(19) United States
(12) Patent Application Publication
(10) Pub. No.: US 2026/0060592 A1
(43) Pub. Date: Mar. 5, 2026

(54) METHODS AND SYSTEMS FOR IDENTIFYING AND ANALYZING HEART RHYTHMS

(71) Applicant: KONINKLIJKE PHILIPS N.V., EINDHOVEN (NL)

(72) Inventors: Wilhelmus Johannes Joseph Stut, Eindhoven (NL); Jan Tatousek, Eindhoven (NL)

(21) Appl. No.: 19/306,009

(22) Filed: Aug. 21, 2025

Related U.S. Application Data
(60) Provisional application No. 63/690,371, filed on Sep. 4, 2024.

(51) Int. Cl. A61B 5/339; A61B 5/346

(52) U.S. Cl. CPC A61B 5/339; A61B 5/346

(57) ABSTRACT

A method for displaying ECG data, comprising: receiving ECG data for a patient for a first time period, comprising: (i) ECG monitoring data and (ii) a plurality of patient-perceived symptoms; generating a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient-perceived symptom, and wherein each of the plurality of columns in the second portion of the symptom matrix comprises a possible ECG event; and displaying, via a user interface, the generated symptom matrix.

### CROSS-REFERENCE TO RELATED APPLICATIONS

This patent application claims the priority benefit under 35 U.S.C. § 119 (c) of U.S. Provisional Application No. 63/690,371, filed on Sep. 4, 2024, the contents of which are herein incorporated by reference.

### FIELD OF THE DISCLOSURE

The present disclosure is directed generally to methods and systems for identifying and analyzing heart rhythms correlating to patient-perceived symptoms utilizing an ECG data display.

### BACKGROUND

The electrocardiogram (ECG) is a non-invasive technique that measures the electrical activity of the heart and is used to detect, among other things, cardiac disorders. Most commonly, ECG measurements are collected in a hospital or in an ambulatory setting. Ambulatory settings typically utilize either a Holter® monitor or mobile cardiac telemetry (MCT). During ambulatory monitoring, the monitor collects, stores, and transmits collected ECG data. Mobile monitoring devices have their advantages and disadvantages. These devices are easier for a patient and physician to apply and maintain, provide the full ECG data for review, and allow for more accurate algorithms to classify arrhythmias. However, for analysis, the patient and/or physician must either mail back the device or upload the data to a server. Additionally, the information collected by the ambulatory ECG monitor is difficult to display in a manner useful to clinicians.

In some embodiments of ambulatory ECG monitoring, the wearer (e.g., the patient or subject) is actively engaged in monitoring by providing an indication of patient-perceived symptoms. For example, the wearer can utilize a reporting mechanism, such as a mobile application, to report when they feel a symptom (e.g. fainting, dizziness, chest pain, etc.), and can describe the symptom(s) and the activity they were engaged in when the symptom(s) occurred. The wearer can select the symptom recording function of the app and can provide details such as the nature of the symptom, the activity during which the symptom occurred (e.g. walking, cycling, sitting, etc.), and/or the (perceived) intensity of the activity (e.g. light, medium, heavy, etc.). In some embodiments the wearer can also press a button on the ECG monitor when they feel a symptom(s).

The data collected by the ambulatory ECG monitor, as well as the patient-perceived symptom reporting, can be collated and displayed to the clinician. However, existing methods of display and analysis are complicated and inadequate. Typically, current methods of display and analysis focus too much on finding and interpreting arrhythmias whereas patient-reported symptoms should form a more important part of the report. In particular, it takes physicians a significant amount of time to identify and analyze the rhythms that occurred when patients felt symptoms.

### SUMMARY OF THE DISCLOSURE

There is thus a continued need for methods and systems that enable the identification and analysis of heart rhythms correlating to patient-perceived symptoms, utilizing an ECG data display.

Various embodiments and implementations are directed to a method and system for displaying ECG data. An ECG analysis or display system received, from an ECG monitor, ECG data comprising ECG monitoring data and patient input regarding a plurality of patient-perceived symptoms. The system generates, from the received ECG data, a symptom matrix comprising a plurality of rows across a first portion and a second portion of the symptom matrix, and further comprising a plurality of columns in each of the first portion and the second portion. Each of the plurality of rows comprises one of the plurality of patient-perceived symptoms. Each of the plurality of columns in the first portion of the symptom matrix comprises a possible patient-perceived symptom, and each of the plurality of columns in the second portion of the symptom matrix comprises a possible ECG event. Each row comprises an indicator in a column in the first portion of the symptom matrix when a patient-perceived symptom corresponds to one of the possible patient-perceived symptoms, and each row comprises an indicator in a column in the second portion of the symptom matrix when a patient-perceived symptom corresponds to one of the possible ECG events. The system displays the generated symptom matrix to a clinician via a user interface.

According to an aspect, a method for displaying ECG data is provided. The method includes receiving, from an ECG monitor, ECG data for a patient for a first time period, wherein the ECG data comprises at least: (i) ECG monitoring data gathered by one or more ECG sensors on the patient's body and (ii) patient input regarding a plurality of patient-perceived symptoms; generating, from the received ECG data for the first time period, a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient-perceived symptom, and wherein each of the plurality of columns in the second portion of the symptom matrix comprises a possible ECG event, wherein each row comprises an indicator in a column in the first portion of the symptom matrix when a patient-perceived symptom corresponds to one of the possible patient-perceived symptoms, and wherein each row comprises an indicator in a column in the second portion of the symptom matrix when a patient-perceived symptom corresponds to one of the possible ECG events; and displaying, via a user interface, the generated symptom matrix.

According to an embodiment, the method further includes receiving, from a user via the user interface, a selection of at least one of the plurality of patient-perceived symptoms; and displaying, via the user interface, an ECG trace associated with the at least one of the plurality of patient-perceived symptoms.

According to an embodiment, the symptom matrix is further adjusted to identify at least one other column in the first portion of the symptom matrix in which the possible patient-perceived symptom in the at least one other column is a patient-perceived symptom in the patient-perceived symptom corresponding to the selected possible patient-perceived symptoms.

According to an embodiment, the method further includes receiving, from a user via the user interface, a selection of at least one of the possible patient-perceived symptoms; and adjusting the symptom matrix to display only the rows in which a patient-perceived symptom corresponds to the selected possible patient-perceived symptoms.

According to an embodiment, the indicator in a column in the second portion of the symptom matrix further comprises ECG data about the possible ECG event corresponding to the patient-perceived symptom.

According to an embodiment, the ECG monitor is a wearable ECG monitor.

According to an embodiment, the patient input is gathered via a smartphone app.

According to an embodiment, the symptom matrix only comprises a column for a possible patient-perceived symptom if that possible patient-perceived symptom corresponds to one of the patient-perceived symptoms in the received ECG data.

According to an embodiment, the plurality of rows each comprising one of the plurality of patient-perceived symptoms are organized by chronological order of the patient-perceived symptoms.

According to an embodiment, the plurality of rows each comprising one of the plurality of patient-perceived symptoms are organized by a severity of the possible ECG events.

According to an embodiment, the symptom matrix further comprises one or more of: (i) a frequency of the patient-perceived symptoms and/or the possible ECG events experienced by the patient; and (ii) a summary of the patient-perceived symptoms and/or the possible ECG events.

According to an embodiment, the method further includes receiving, from an ECG monitor, ECG data for the patient for a second time period, wherein the ECG data comprises at least: (i) ECG monitoring data gathered by one or more ECG sensors on the patient's body and (ii) patient input regarding a plurality of patient-perceived symptoms; and generating a trend graph showing a summary of the plurality patient-perceived symptoms for the first time period and the plurality patient-perceived symptoms for the second time period.

According to another aspect is a system for displaying ECG data. The system includes ECG data obtained from an ECG monitor for a patient for a first time period, wherein the ECG data comprises at least: (i) ECG monitoring data gathered by one or more ECG sensors on the patient's body and (ii) patient input regarding a plurality of patient-perceived symptoms; a processor configured to generate, from the received ECG data for the first time period, a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient-perceived symptom, and wherein each of the plurality of columns in the second portion of the symptom matrix comprises a possible ECG event, wherein each row comprises an indicator in a column in the first portion of the symptom matrix when a patient-perceived symptom corresponds to one of the possible patient-perceived symptoms, and wherein each row comprises an indicator in a column in the second portion of the symptom matrix when a patient-perceived symptom corresponds to one of the possible ECG events; and a user interface configured to display the generated symptom matrix.

According to another aspect is a system for displaying ECG data. The system includes an ambulatory ECG device configured to obtain, for a first time period, ECG monitoring data gathered by one or more ECG sensors on a patient's body; a computerized device configured to obtain input from the patient regarding a plurality of patient-perceived symptoms during the first time period; a processor remote from the ambulatory ECG device and the computerized device, the processor configured to generate, from both the received ECG monitoring data for the first time period and the plurality of patient-perceived symptoms during the first time period, a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient-perceived symptom, and wherein each of the plurality of columns in the second portion of the symptom matrix comprises a possible ECG event, wherein each row comprises an indicator in a column in the first portion of the symptom matrix when a patient-perceived symptom corresponds to one of the possible patient-perceived symptoms, and wherein each row comprises an indicator in a column in the second portion of the symptom matrix when a patient-perceived symptom corresponds to one of the possible ECG events; and a user interface configured to display the generated symptom matrix.

According to an embodiment, the symptom matrix further comprises one or more of: (i) a frequency of the patient-perceived symptoms and/or the possible ECG events experienced by the patient; and (ii) a summary of the patient-perceived symptoms and/or the possible ECG events experienced by the patient.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for identifying and analyzing heart rhythms correlating to patient-perceived symptoms utilizing an ECG data display, in accordance with an embodiment.

FIG. 2 is a schematic representation of an ECG device, in accordance with an embodiment.

FIG. 3 is a schematic representation of an ECG data analysis and display system, in accordance with an embodiment.

FIG. 4 is a schematic representation of a patient symptom reporting device, in accordance with an embodiment.

FIG. 5 is a schematic representation of a symptom matrix, in accordance with an embodiment.

FIG. 6 is a schematic representation of a symptom matrix, in accordance with an embodiment.

FIG. 7 is a schematic representation of a symptom matrix, in accordance with an embodiment.

FIG. 8 is schematic representation of an ECG trend graph, in accordance with an embodiment.

FIG. 9 is a schematic representation of a symptom matrix, in accordance with an embodiment.

### DETAILED DESCRIPTION OF EMBODIMENTS

The present disclosure describes various embodiments of a system and method configured to identify and analyze heart rhythms correlating to patient-perceived symptoms utilizing an ECG data display. More generally, Applicant has recognized and appreciated that it would be beneficial to provide a method and system to overcome the limitations of analyzing and identifying heart rhythms using existing methods. An ECG analysis or display system received, from an ECG monitor, ECG data comprising ECG monitoring data and patient input regarding a plurality of patient-perceived symptoms. The system generates, from the received ECG data, a symptom matrix comprising a plurality of rows across a first portion and a second portion of the symptom matrix, and further comprising a plurality of columns in each of the first portion and the second portion. Each of the plurality of rows comprises one of the plurality of patient-perceived symptoms. Each of the plurality of columns in the first portion of the symptom matrix comprises a possible patient-perceived symptom, and each of the plurality of columns in the second portion of the symptom matrix comprises a possible ECG event. Each row comprises an indicator in a column in the first portion of the symptom matrix when a patient-perceived symptom corresponds to one of the possible patient-perceived symptoms, and each row comprises an indicator in a column in the second portion of the symptom matrix when a patient-perceived symptom corresponds to one of the possible ECG events. The system displays the generated symptom matrix to a clinician via a user interface.

The embodiments and implementations disclosed or otherwise envisioned herein can be utilized with any ECG system or process that may utilize or benefit from improved identification and analysis of heart rhythms correlating to patient-perceived symptoms utilizing an ECG data display. For example, the systems and methods described or otherwise envisioned herein can, in some non-limiting embodiments, be implemented as an element for a commercial product for patient analysis or monitoring, such as Philips® ECG systems and devices (available from Koninklijke Philips NV, the Netherlands), or any other suitable system. However, the disclosure is not limited to these devices or systems, and thus disclosure and embodiments disclosed herein can encompass any system that may utilize or benefit from identification and analysis of heart rhythms correlating to patient-perceived symptoms utilizing an ECG data display.

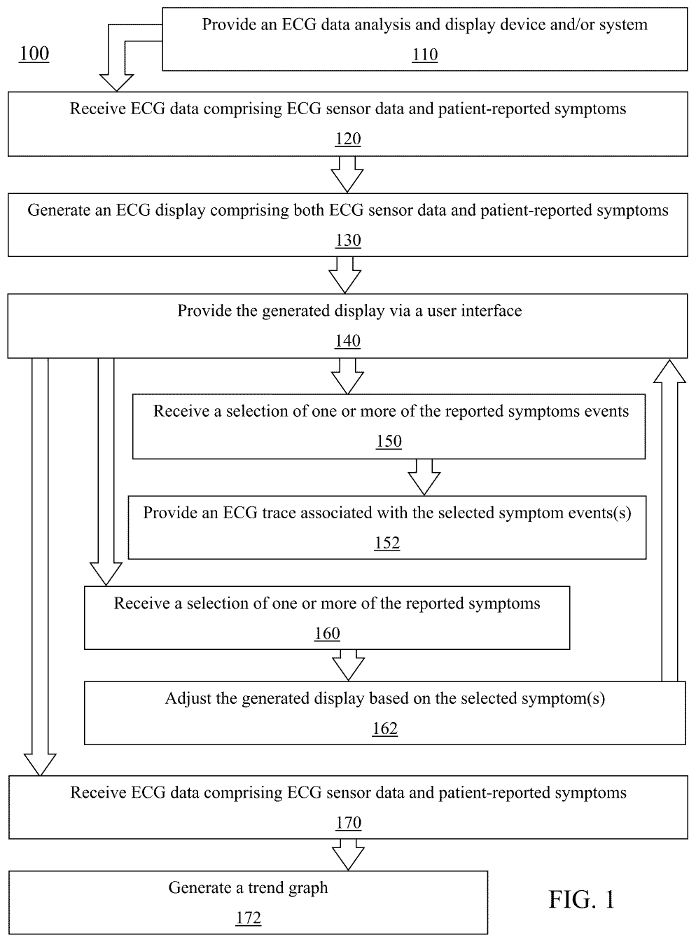

Referring to FIG. 1, in one embodiment, is a flowchart of a method 100 for displaying ECG data using an ECG data analysis and display system. The methods described in connection with the figures are provided as examples only, and shall be understood not to limit the scope of the disclosure. The ECG data analysis and display system can be any of the devices or systems described or otherwise envisioned herein. The ECG data analysis and display system can be a single device or system, or can be multiple different devices or systems.

At step 110 of the method, an ambulatory ECG device 200 and an ECG data analysis and display system 300 is provided.

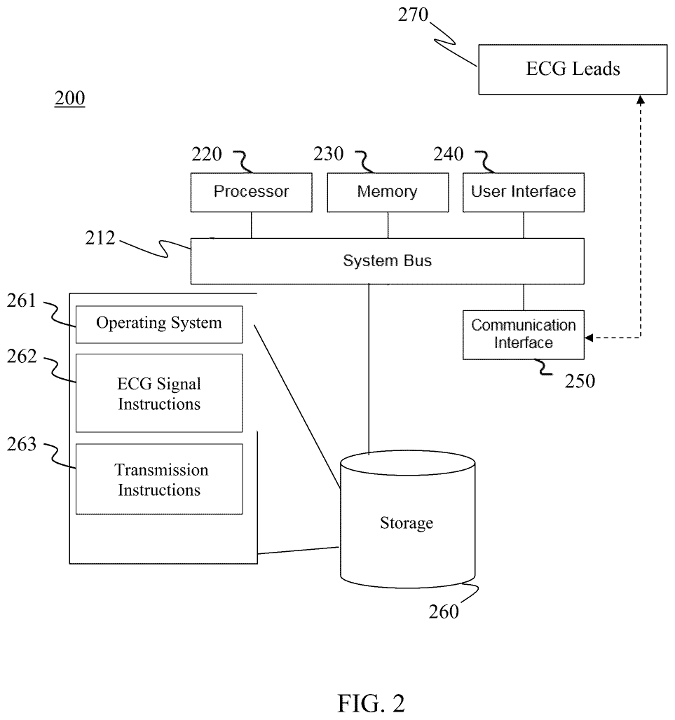

Referring to an embodiment of an ambulatory ECG device 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the ambulatory ECG device 200 may be different and more complex than illustrated. Additionally, ambulatory ECG device 200 can be any of the devices described or otherwise envisioned herein. Other elements and components of the ambulatory ECG device 200 are disclosed and/or envisioned elsewhere herein.

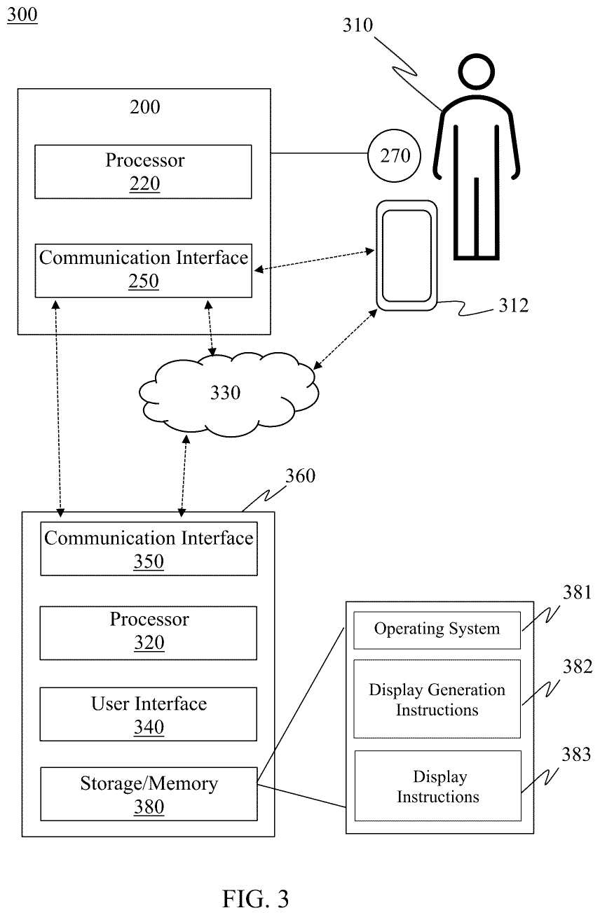

According to an embodiment, the ambulatory ECG device 200 comprises or is in direct or indirect communication with one or more ECG leads 270 configured to obtain ECG data from a subject (as shown in FIG. 3). Accordingly, the one or more ECG leads 270 will be positioned in contact with the subject, according to known methods, and can obtain ECG data from that subject. Once obtained, the ECG data may be utilized immediately, and/or it may be temporarily or permanently stored in memory for future use.

Referring to an embodiment of an ECG data analysis and display system 300 as depicted in FIG. 3, for example, the system comprises one or more of an ambulatory ECG device 200, which according to an embodiment is an ambulatory ECG device, which comprises one or more ECG leads 270 configured to obtain an ECG signal from a subject 310. ECG data analysis and display system 300 further comprises a remote server or computer 360, which receives ECG data from the ECG device 200. For example, the ECG data can be transmitted by the communication interface 250 of the ECG device to the communication interface 350 of the remote server or computer 360, either directly or through a communications network 330. According to an embodiment, system 300 further comprises a smartphone 312 or other computerized device with a reporting application via which subject 310 can report patient-perceived symptoms or other events relevant to the ECG monitoring. The computerized device 312 can transmit the patient-reported information to the ECG device 200, or can communicate the patient-reported information to the communication interface 350 of the remote server or computer 360, such as via a communications network 330.

Thus, according to an embodiment, the ECG data received by the remote server or computer 360 comprises both the ECG monitoring data from the ambulatory ECG device 200 and reported patient-perceived symptom information from the computerized device 312. Once received, the ECG data can be stored in memory, and/or can be analyzed by a processor 320 of the remote server or computer 360. The remote server or computer 360 further comprises or is in direct or indirect communication with a user interface 340 that can be utilized to display one or more aspects of the ECG data as described or otherwise envisioned herein.

At step 120 of the method, the ECG data analysis and display system 300 receives, requests, or otherwise obtains ECG data comprising both the ECG monitoring data from the ambulatory ECG device 200 and reported patient-perceived symptom information from the computerized device 312. The ECG device may obtain an ECG signal using known methods for obtaining ECG signals. The signal may be obtained continuously or periodically as determined by the settings or parameters of the ECG device or system. When the ECG device is an ambulatory device, the ECG signal may be obtained while the subject is ambulatory. Thus, according to an embodiment, “ambulatory” can mean that the subject is utilizing a mobile ECG device outside a clinical setting, rather than using a stationary or mobile ECG device within a clinical setting. Once obtained, the ECG signal may be utilized immediately and/or it may be saved in memory for future analysis.

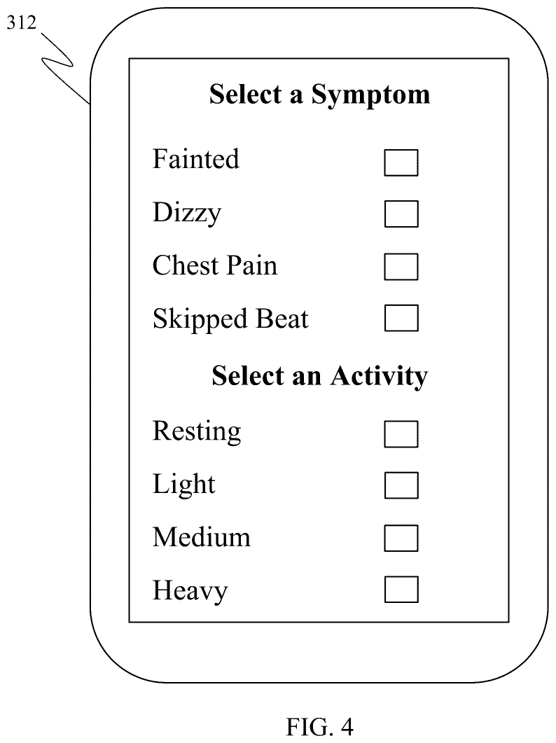

According to an embodiment, the system receives reported patient-perceived symptom information from the computerized device 312. The computerized device 312 may collect or obtain or receive the patient-perceived symptom information in a variety of mechanisms. For example, the device may comprise an application specifically designed to collect and report patient-perceived symptom information, among other possible mechanisms. Referring to FIG. 4, in accordance with one non-limiting embodiment, is a smartphone or other computerized device 312 in which the user can report patient-perceived symptoms, which can optionally include a patient activity. The device 312 can comprise an application or other programming to facilitate the collection and sharing of patient-perceived symptom information. When the subject wearing the ambulatory ECG device 200 feels a symptom, they use device 312 to describe the symptom(s) and the activity they were engaged in when the symptom(s) occurred. They select the symptom recording function and provide details like the nature of the symptom (e.g. fainting, dizziness, chest pain, etc.), the activity during which the symptom occurred (e.g. walking, cycling), and/or the (perceived) intensity of the activity (e.g. resting, light, medium, heavy, etc.). In some embodiments patients can also press a button on the ECG monitor when they feel symptoms.

Once obtained, the ECG data comprising both the ECG monitoring data from the ambulatory ECG device 200 and reported patient-perceived symptom information from the computerized device 312 may be utilized immediately and/or it may be saved in memory for future analysis.

At step 130 of the method, a component of the ECG data analysis and display system 300 analyzes the received ECG data to generate an ECG display comprising both the ECG monitoring data from the ambulatory ECG device 200 and reported patient-perceived symptom information from the computerized device 312. According to an embodiment, the ECG data is analyzed by processor 220 of the ECG device 200, and/or by processor 320 of the receiving remote server or computer 360.

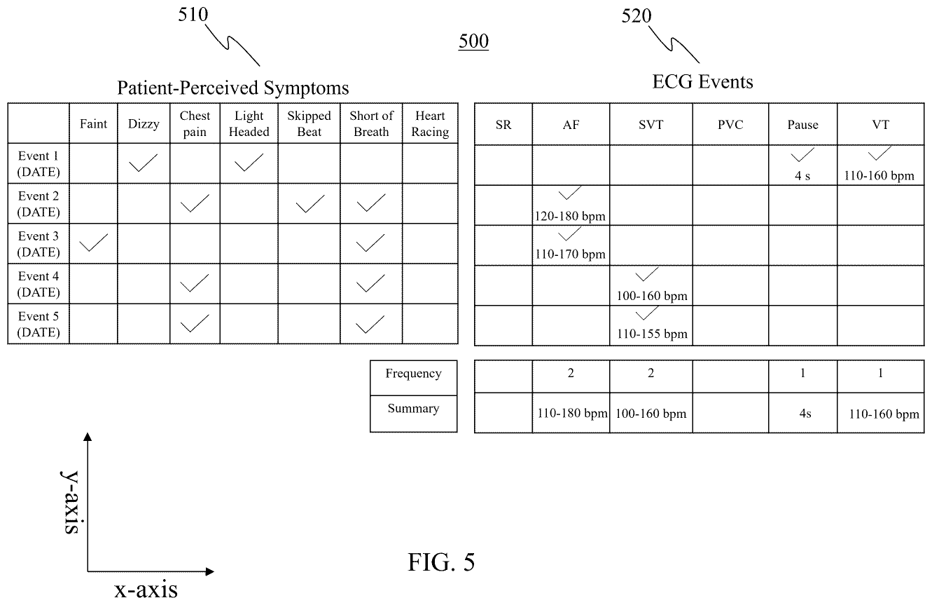

According to an embodiment, the ECG display generated by the system comprises a symptom matrix with the ECG data and reported patient-perceived symptom information. Referring to FIG. 5, in one embodiment, is a non-limiting example of a generated symptom matrix 500. The symptom matrix 500 comprises a first portion 510 comprising patient-perceived symptom information, and a second portion 520 comprising ECG monitoring data. The first and second portions may be separated or joined.

According to an embodiment, the symptom matrix 500 further comprises a plurality of rows (oriented along an x-axis) and a plurality of columns (oriented perpendicular along a y-axis) in each of the first portion and the second portion.

According to an embodiment, the plurality of rows in the first portion and the second portion of the symptom matrix comprises one of a plurality of patient-perceived symptoms. For example, referring to FIG. 5, there are five (5) patient-perceived symptoms (“Event 1”, “Event 2,” etc.) which may represent five events reported and recorded during a monitoring time period (i.e., “a first time period”). The patient-perceived symptom may be accompanied with other information such as the date and/or time of the event.

According to an embodiment, each of the columns in the first portion of the symptom matrix represent a possible patient symptom. For example, possible symptoms include “Faint,” “Dizzy,” “Chest pain,” “Lightheaded,” “Skipped Beat,” “Short of Breath,” “Heart Racing,” and/or other possible symptoms. According to an embodiment, each of the columns in the second portion of the symptom matrix represent a possible ECG event, such as SR (sinus rhythm), AF (atrial fibrillation), SVT (supraventricular tachycardia), PVC (premature ventricular contraction), Pause, VT (ventricular tachycardia), and/or other possible ECG events. Although FIG. 5 only shows certain possible patient symptoms and certain possible ECG events, many others are possible.

The symptom matrix comprises individual cells where the rows and columns intersect. According to an embodiment, a cell comprises an indicator when there is content that is found in both the intersecting row and the intersecting column. For example, referring to FIG. 5, Event 1 comprises several reported patient symptoms including dizziness and lightheadedness, and several ECG events including a pause of 4 seconds and ventricular tachycardia with 110-160 bpm. Thus, a row comprises an indicator in a column in the first portion of the symptom matrix when a patient-perceived symptom corresponds to one of the possible patient symptoms. Similarly, a row comprises an indicator in a column in the second portion of the symptom matrix when a patient-perceived symptom corresponds to one of the possible ECG events.

According to an embodiment, an indicator can be anything that indicates an intersection of a patient symptom reported by the patient and an ECG event detected by the ECG device. For example, referring to FIG. 5, the indicator is a checkmark. However, many other indicators are possible. The indicator may be a text-based or image-based indicator, such as a checkmark, an “x,” an icon, or any other indicator. The indicator may be another type of indicator such as a highlighted cell, a colored cell, or any other indicator.

According to an embodiment, a cell may comprise additional information. For example, referring to FIG. 5, the cells comprising an indicator in the second portion 520 of the symptom matrix comprise additional information. The cell for “Pause” in the row for “Event 1” comprises “4 s” indicating a pause of four seconds. The cell for ventricular tachycardia (VT) in the row for “Event 1” comprises additional information “110-160 bpm” indicating the range for beats per minute recorded during the event. Thus, the symptom matrix may communicate characteristics of the arrhythmias, such as the minimum and maximum heart rate during the event. Depending on the arrhythmia, different characteristics may be shown. For example, for Pause the duration can be mentioned. Many other variations are possible.

Although not shown in FIG. 5, the cells in the first portion 510 (and, optionally, the second portion 520) of the symptom matrix with an indicator could include additional information such as activity information, which is optionally collected by the computerized device 312. For example, a cell may comprise highlighting, text, or coloring that indicates a reported activity level at the time of the patient-perceived symptom. As just one non-limiting example, the cell indicating “Dizzy” for Event 1 may be colored red to indicate a high level of activity at the time of the Event. Additionally or alternatively, the entire row for Event 1 may be colored or otherwise comprise an indicator revealing the level of activity at the time of the event.

According to an embodiment, the symptom matrix further comprises summary information for the possible patient symptoms reported by the patient and/or the possible ECG events detected by the ECG device. For example, referring to FIG. 5, the symptom matrix comprises “Frequency” and “Summary” rows containing summary information for the ECG event columns. The “AF” column contains two events, so the frequency row comprises an indicator of “2” and the summary row comprises the detected range of 110-180 bpm. Thus, for example, for two patient-reported events (Event 2 and Event 3) an AF was detected, with a minimum heart rate during these two events of 110 and a maximum heart rate of 180.

At step 140 of the method, the generated symptom matrix is provided to a viewer, user, clinician, or other individual via a user interface. The user interface can be any user interface of the ECG data analysis and display system 300.

According to an embodiment, the symptom matrix is interactive. For example, a portion of the symptom matrix may be clickable or otherwise engaged with, via the user interface on which the symptom matrix is displayed, to result in an action. A row, a column, and/or a cell may be clickable. Thus, a row, a column, and/or a cell may be clickable to show, share, report, or otherwise indicate an ECG trace associated with the row, column, and/or cell. The clinician may click or otherwise interact with Event 1—such as clicking the row for Event 1, or clicking the reported symptoms or ECG event cells in the row for Event 1—to result in the ECG trace associated with Event 1 to show, pop-up, or otherwise be shared or reviewable.

Accordingly, at step 150 of the method, the ECG data analysis and display system 300 receives a selection from a viewer, user, clinician, or other individual, via a user interface, of at least one of the plurality of patient-perceived symptoms. At step 152 of the method, the system generates a symptom matrix, or adjusts or regenerates the previous symptom matrix, to show an ECG trace associated with the selected at least one of the plurality of patient-perceived symptoms.

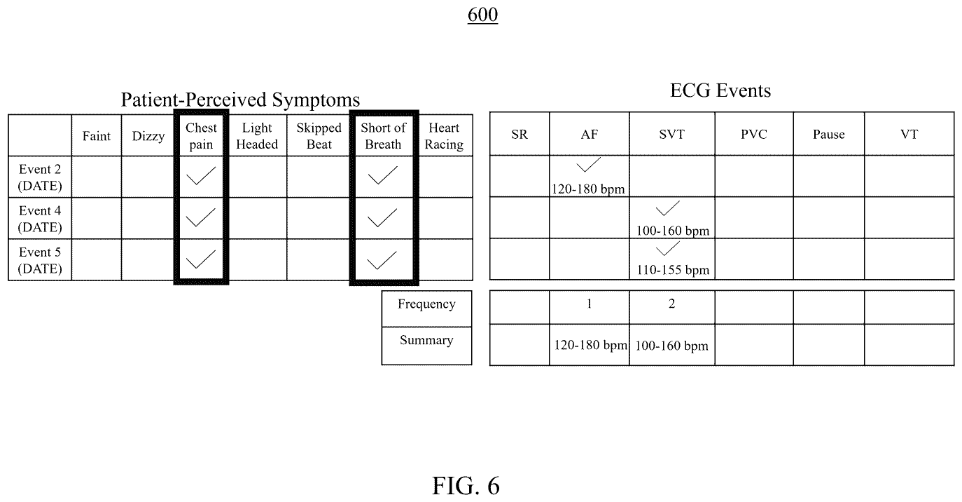

According to another embodiment of an interactive symptom matrix, the clinician can focus on patient-perceived symptoms by selecting one or more of the symptom columns in the first portion 510. To focus on one or more symptoms, the clinician can select these symptoms, e.g. by ticking a check box or otherwise selecting a column, row, or cell. For example, when the clinician selects the “Chest Pain” symptom, the symptom matrix will adjust to who only the events with that symptom. Referring to FIG. 6, for example, the symptom matrix is only showing Events 2, 4, and 5 as they are the events with a reported “Chest Pain” symptom. In this embodiment, the other events (i.e., Event 1 and 3) are removed, although other mechanisms are possible.

According to an embodiment, the symptom matrix can automatically highlight symptoms that occurred together with the selected symptom(s) during the ambulatory ECG study for the first time period. For example, referring to FIG. 5, the clinician has selected “Chest pain” and every time the subject felt chest pain, the subject felt short of breath and thus the column for “Short of Breath” is similarly highlighted. According to an embodiment, the additional information (such as “Frequency” and “Summary”) is also adjusted to reflect the selected rows. In this embodiment, the additional information shows 1 AF and 2 SVTs. Note that in this case the findings and their characteristic, can be used to understand the similarities and/or differences between the patient-reported events with the same symptom(s). This comparison can be done by the physician or by AI-powered software

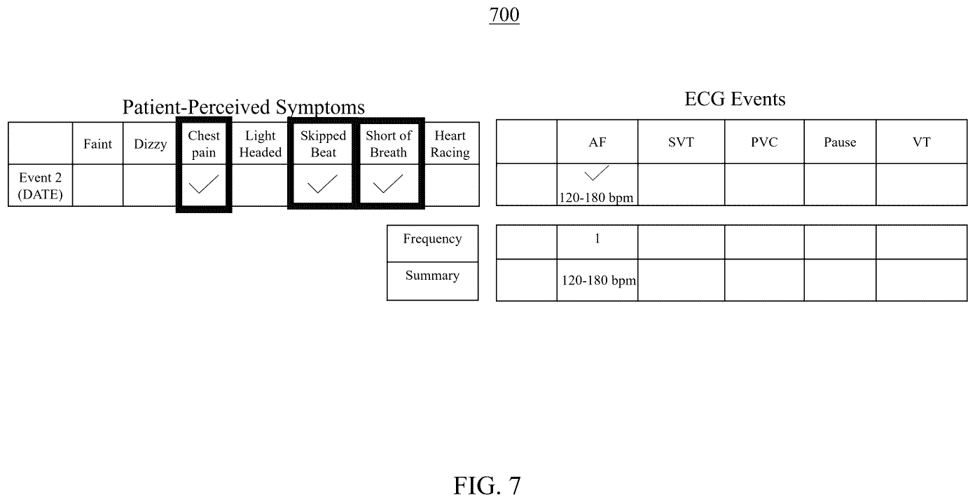

According to another embodiment of an interactive symptom matrix, the clinician can focus on patient-perceived symptoms by selecting two or more of the symptom columns in the first portion 510. The clinician may select multiple symptoms, so that only the events with all selected symptoms are shown. In the example shown in FIG. 7, the clinician has selected both “Chest pain” and “Skipped beat,” and it also shows “short of breath.” When clicking on this event, the corresponding ECG trace will be shown.

According to an embodiment, instead of only using AND semantics, one could add an event search builder like the way PubMed allows to use logical operators for finding public. For example, Chest Pain AND Skipped Beat AND NOT Dizzy.

Accordingly, at step 160 of the method, the ECG data analysis and display system 300 receives a selection from a viewer, user, clinician, or other individual, via a user interface, of at least one of the possible patient symptoms. At step 162 of the method, the system generates a symptom matrix, or adjusts or regenerates the previous symptom matrix, to show the rows in which a patient-perceived symptom corresponds to the selected possible patient symptoms.

According to an embodiment, the system may show symptom trends over time. At step 170 of the method, the ECG data analysis and display system 300 receives, requests, or otherwise obtains ECG data comprising both the ECG monitoring data from the ambulatory ECG device 200 and reported patient-perceived symptom information from the computerized device 312, for a second time period different from the first time period. The ECG device may obtain an ECG signal using known methods for obtaining ECG signals, as described or otherwise envisioned herein. The computerized device 312 may collect or obtain or receive the patient-perceived symptom information in a variety of mechanisms, as described or otherwise envisioned herein.

Once obtained, the ECG data comprising both the ECG monitoring data from the ambulatory ECG device 200 and reported patient-perceived symptom information from the computerized device 312 may be utilized immediately and/or it may be saved in memory for future analysis.

At step 172 of the method, the system generates a trend graph showing a summary of the plurality patient-perceived symptoms for the first time period and the plurality patient-perceived symptoms for the second time period.

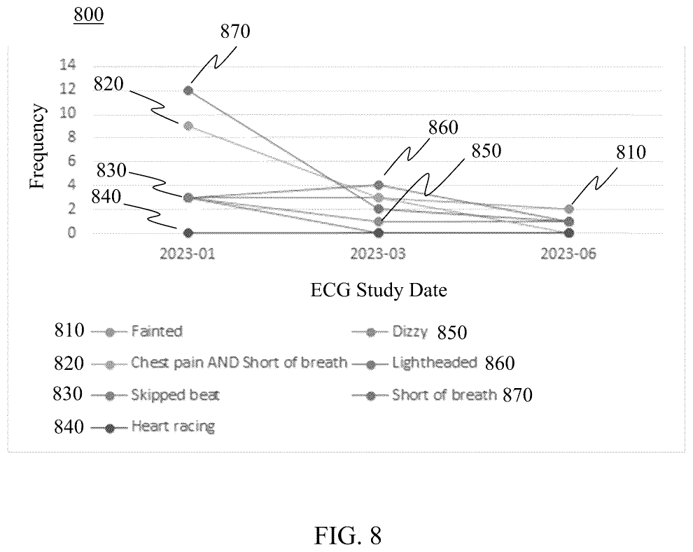

For example, referring to FIG. 8, is a trend graph 800 showing a summary of the plurality patient-perceived symptoms for three time periods (2023-01, 2023-03, and 2023-06). The graph shows the number of times a symptom (or combination thereof) occurred during these three ECG studies. For example, Short of breath (870) occurred 12 times in the study of January 2023, and (responsive to an intervention in February 2023) only 2 times in the study of March 2023. The symptoms trend graph can show the symptom frequency per study (e.g., 2 weeks), but can also show the symptom frequency per time interval (e.g., per day, week, or month). This could be especially useful for handheld ECG monitors and longer-term monitors such as implantable loop recorders and wearables such as a watch or other wearable.

According to an embodiment, hoovering over a data point in the trend graph shows the characteristics of the findings around the patient-reported symptoms that are associated with that data point. The dashboard could also generate and show an interpretation and treatment suggestion. For example, it could suggest starting a particular medication for treating the combination of fainting with AF and SVT.

According to an embodiment, regarding a certain datapoint it is possible to show the findings for that specific timestep but maybe also compute some high-level preliminary analysis. This can be accomplished by leveraging computation on the QRS complex, so average HR is one. The system could also personalize the view by including HRV, PR interval, PR segment, ST segment, QT interval, QRS complex interval.

According to an embodiment, the system could allow for an alarm when one of the values exceed a limit in combination with a registered symptom, as it might require some adjustments in the medication. This could function in background and simply raise a notification at the next follow up or if more urgent immediately.

Other embodiments of the symptom matrix and system are possible. For example, the symptom matrix could be generated to show only the symptoms that occurred in the ambulatory ECG study during the first time period. The symptom matrix could be generated to show only the symptoms that occurred in this and other ambulatory ECG studies of this patient. The symptom matrix could be generated to show only the symptoms that occur in patients with a similar medical condition and/or history.

According to an embodiment, the symptom matrix could be generated to show the possible patient symptoms in alphabetical order, or ordered by symptom frequency (decreasing or increasing) in the ambulatory ECG study of the first time period, or ordered by symptom frequency in this and/or other ambulatory ECG studies of this patient, or ordered by symptom frequency ordered by symptom frequency in the population or a subset thereof (e.g. males aged 50-65 years), among other mechanisms.

According to an embodiment, the symptom matrix could be generated to show the possible ECG events ordered by number of symptoms per event (decreasing or increasing), or ordered by severity of findings (e.g. events with life-threatening arrhythmias on top), among other mechanisms.

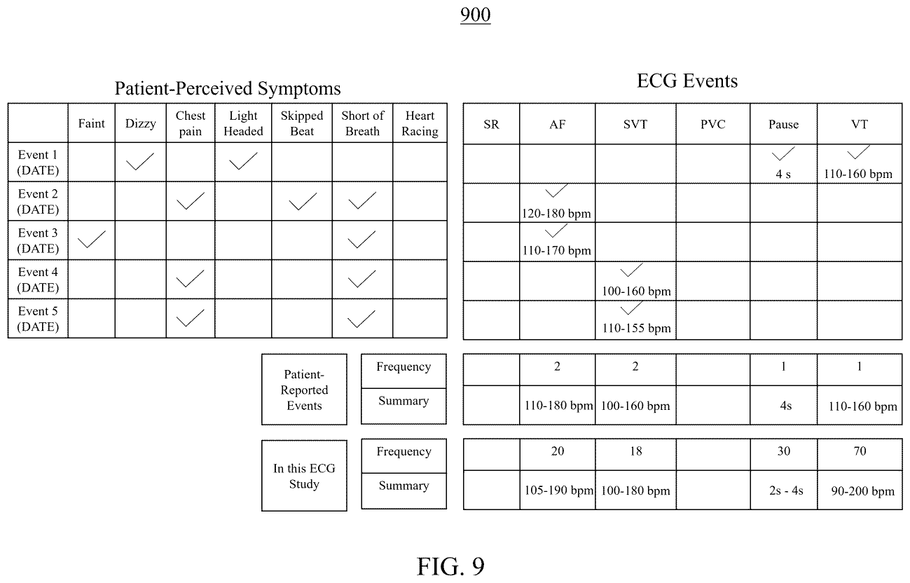

According to an embodiment, in addition to showing the frequency and summary of the findings associated with patient-reported events, the dashboard can also show the frequency and summary of all findings in the ambulatory ECG study (or studies of this patient). For example, referring to FIG. 9, the summary portion comprises both an ECG event frequency and ECG event summary associated with the patient-perceived symptoms, and an ECG event frequency and ECG event summary associated the entire ECG study (i.e., for the first time period).

Referring again to FIG. 2 is a schematic representation of an ECG device 200. Device 200 may be any of the devices or systems described or otherwise envisioned herein, and may comprise any of the components described or otherwise envisioned herein. It will be understood that FIG. 2 constitutes, in some respects, an abstraction and that the actual organization of the components of device 200 may be different and more complex than illustrated.

According to an embodiment, device 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While device 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of device 200 is implemented in a cloud computing system, the various hardware components may belong to separate physical systems. For example, processor 220 may include a first processor in a first server and a second processor in a second server. Many other variations and configurations are possible.

According to an embodiment, storage 260 of device 200 may store one or more algorithms, modules, and/or instructions to carry out one or more functions or steps of the methods described or otherwise envisioned herein. For example, storage 260 may comprise, among other instructions or data, ECG signal instructions 262, and/or transmission instructions 263.

According to an embodiment, ECG signal instructions 262 direct the system to obtain ECG data from a patient. The ECG device may obtain an ECG signal using known methods for obtaining ECG signals. The signal may be obtained continuously or periodically as determined by the settings or parameters of the ECG device or system. When the ECG device is an ambulatory device, the ECG signal may be obtained while the subject is ambulatory. Thus, according to an embodiment, “ambulatory” can mean that the subject is utilizing a mobile ECG device outside a clinical setting, rather than using a stationary or mobile ECG device within a clinical setting. Once obtained, the ECG signal may be utilized immediately and/or it may be saved in memory for future analysis.

According to an embodiment, transmission instructions 263 direct the system to transmit the compressed ECG signal. The compressed ECG signal may be transmitted by the communication interface 250 of the ECG device. The transmission may be wired and/or wireless, directly and/or via a communications network. According to an embodiment, the ambulatory ECG device transmits the compressed ECG signal continuously or periodically. The ambulatory ECG device may transmit the compressed ECG signal pursuant to a programmed schedule and/or in response to a command or query. The command or query may be made by a human such as a patient or physician, or the command or query may be made by another local or remote device or system.

Referring to FIG. 3, in one embodiment, an ECG data analysis and display system 300 is provided. Referring to an embodiment of an ECG data analysis and display system 300 as depicted in FIG. 3, for example, the system comprises one or more of an ECG device 200, which according to an embodiment is an ambulatory ECG device, which comprises one or more ECG leads 270 configured to obtain an ECG signal from a subject 310. System 300 further comprises a remote server or computer 360, which receives a compressed ECG signal from the ECG device 200. For example, the compressed ECG signal can be transmitted by the communication interface 250 of the ECG device to the communication interface 350 of the remote server or computer 360, either directly or through a communications network 330. Once received, the compressed ECG signal can be stored in memory, and/or can be analyzed by a processor 320 of the remote server or computer 360 to generate a reconstructed ECG signal. The remote server or computer 360 further comprises or is in direct or indirect communication with a user interface 340 that can be utilized to display one or more aspects of a reconstructed ECG signal.

According to an embodiment, remote server or computer 360 further comprises storage or memory 360. Storage 260 may include one or more machine-readable storage media such as read-only memory (ROM), random-access memory (RAM), magnetic disk storage media, optical storage media, flash-memory devices, or similar storage media. In various embodiments, storage 380 may store instructions for execution by processor 320 or data upon which processor 320 may operate. For example, storage 380 may store an operating system 381 for controlling various operations of system 300. Storage 380 may be considered to be non-transitory machine-readable media. As used herein, the term non-transitory will be understood to exclude transitory signals but to include all forms of storage, including both volatile and non-volatile memories.

According to an embodiment, storage 380 of system 300 may store one or more algorithms, modules, and/or instructions to carry out one or more functions or steps of the methods described or otherwise envisioned herein. For example, storage 380 may comprise, among other instructions or data, display generation instructions 382, and/or display or reporting instructions 383.

According to an embodiment, display generation instructions 382 direct the system to generate, from received ECG data comprising both the ECG monitoring data from the ambulatory ECG device 200 and reported patient-perceived symptom information from the computerized device 312. The ECG device may obtain an ECG signal using known methods for obtaining ECG signals, as described or otherwise envisioned herein. The computerized device 312 may collect or obtain or receive the patient-perceived symptom information in a variety of mechanisms, as described or otherwise envisioned herein. According to an embodiment, the ECG display generated by the system comprises a symptom matrix with the ECG data and reported patient-perceived symptom information, according to any of the embodiments or methods envisioned or described herein.

According to an embodiment, display or reporting instructions 384 direct the system to provide the output of the system-such as the generated symptom matrix—to a patient, clinician, or other device or system. The provided output can be any of the information as described or otherwise envisioned herein. The system may provide the information to a user via any mechanism, including but not limited to a visual display, an audible notification, a page, or any other method of notification. The information may be communicated by wired and/or wireless communication to another device. For example, the system may communicate the information to a monitor, screen, mobile phone, computer, laptop, wearable device, and/or any other device configured to allow display and/or other communication of the information. According to an embodiment, the system displays some or all of the generated symptom matrix via a user interface of the system, such as user interface 240 and/or user interface 340.

The generation and display of the described symptom matrix, as recited in the claims (e.g., a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient symptom, and wherein each of the plurality of columns in the second portion of the symptom matrix comprises a possible ECG event) represents a significant technological improvement over the prior art in at least two different ways.

First, the implementation of the system to generate the symptom matrix provides an improvement to the underlying computing device. For example, by intelligently managing graphical elements and rendering processes (to generate the symptom matrix), the software reduces the computational load of the computing device, thereby enhancing the overall performance and responsiveness of the computing device. In contrast, prior art systems only display a minute portion of the reported patient-perceived symptoms, if any. Accordingly, these systems require considerable additional computational effort to generate a report of multiple or all patient-perceived symptoms, since these prior art systems are not programmed to generate or provide this information. This is in stark contrast to the streamlined symptom matrix recited in the claims and described in the specification. Thus, implementation of the system to generate the symptom matrix is a significant technological improvement over the prior art.

Second, improved understanding of the reported patient-perceived symptoms during an ambulatory ECG study (or multiple studies), together with the underlying ECG events and/or ECG traces, significantly improves clinician review of the ambulatory ECG study and patient diagnosis. Having a fuller understanding of reported patient-perceived symptoms and associated ECG events —which is in stark contrast to prior art systems that provide very few, if any, reported patient-perceived symptoms-allows for a faster and more complete analysis and diagnosis of the ambulatory ECG study.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for displaying ECG data, comprising:
receiving, from an ECG monitor, ECG data for a patient for a first time period, wherein the ECG data comprises at least: (i) ECG monitoring data gathered by one or more ECG sensors on the patient's body and (ii) patient input regarding a plurality of patient-perceived symptoms;
generating, from the received ECG data for the first time period, a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient-perceived symptom, and wherein each of the plurality of columns in the second portion of the symptom matrix comprises a possible ECG event, wherein each row comprises an indicator in a column in the first portion of the symptom matrix when a patient-perceived symptom corresponds to one of the possible patient-perceived symptoms, and wherein each row comprises an indicator in a column in the second portion of the symptom matrix when a patient-perceived symptom corresponds to one of the possible ECG events; and
displaying, via a user interface, the generated symptom matrix.

2. The method of claim 1, further comprising:
receiving, from a user via the user interface, a selection of at least one of the plurality of patient-perceived symptoms; and
displaying, via the user interface, an ECG trace associated with the at least one of the plurality of patient-perceived symptoms.

3. The method of claim 2, wherein the symptom matrix is further adjusted to identify at least one other column in the first portion of the symptom matrix in which the possible patient-perceived symptom in the at least one other column is a patient-perceived symptom in the patient-perceived symptom corresponding to the selected possible patient-perceived symptoms.

4. The method of claim 1, further comprising:
receiving, from a user via the user interface, a selection of at least one of the possible patient-perceived symptoms; and
adjusting the symptom matrix to display only the rows in which a patient-perceived symptom corresponds to the selected possible patient-perceived symptoms.

5. The method of claim 1, wherein the indicator in a column in the second portion of the symptom matrix further comprises ECG data about the possible ECG event corresponding to the patient-perceived symptom.

6. The method of claim 1, wherein the ECG monitor is a wearable ECG monitor.

7. The method of claim 1, wherein the patient input is gathered via a smartphone app.

8. The method of claim 1, wherein the symptom matrix only comprises a column for a possible patient-perceived symptom if that possible patient-perceived symptom corresponds to one of the patient-perceived symptoms in the received ECG data.

9. The method of claim 1, wherein the plurality of rows each comprising one of the plurality of patient-perceived symptoms are organized by chronological order of the patient-perceived symptoms.

10. The method of claim 1, wherein the plurality of rows each comprising one of the plurality of patient-perceived symptoms are organized by a severity of the possible ECG events.

11. The method of claim 1, wherein the symptom matrix further comprises one or more of: (i) a frequency of the patient-perceived symptoms and/or the possible ECG events experienced by the patient; and (ii) a summary of the patient-perceived symptoms and/or the possible ECG events.

12. The method of claim 1, further comprising:
receiving, from an ECG monitor, ECG data for the patient for a second time period, wherein the ECG data comprises at least: (i) ECG monitoring data gathered by one or more ECG sensors on the patient's body and (ii) patient input regarding a plurality of patient-perceived symptoms; and
generating a trend graph showing a summary of the plurality patient-perceived symptoms for the first time period and the plurality patient-perceived symptoms for the second time period.

13. A system for displaying ECG data, comprising:
ECG data obtained from an ECG monitor for a patient for a first time period, wherein the ECG data comprises at least: (i) ECG monitoring data gathered by one or more ECG sensors on the patient's body and (ii) patient input regarding a plurality of patient-perceived symptoms;
a processor configured to generate, from the received ECG data for the first time period, a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient-perceived symptom, and wherein each of the plurality of columns in the second portion of the symptom matrix comprises a possible ECG event, wherein each row comprises an indicator in a column in the first portion of the symptom matrix when a patient-perceived symptom corresponds to one of the possible patient-perceived symptoms, and wherein each row comprises an indicator in a column in the second portion of the symptom matrix when a patient-perceived symptom corresponds to one of the possible ECG events; and
a user interface configured to display the generated symptom matrix.

14. A system for displaying ECG data, comprising:
an ambulatory ECG device configured to obtain, for a first time period, ECG monitoring data gathered by one or more ECG sensors on a patient's body;
a computerized device configured to obtain input from the patient regarding a plurality of patient-perceived symptoms during the first time period;
a processor remote from the ambulatory ECG device and the computerized device, the processor configured to generate, from both the received ECG monitoring data for the first time period and the plurality of patient-perceived symptoms during the first time period, a symptom matrix comprising a plurality of rows across a first portion and a second portion of the symptom matrix, and further comprising a plurality of columns in each of the first portion and the second portion, wherein each of the plurality of rows comprises one of the plurality of patient-perceived symptoms, and wherein each of the plurality of columns in the first portion of the symptom matrix comprises a possible patient-perceived symptom, and wherein each of the plurality of columns in the second portion of the symptom matrix comprises a possible ECG event, wherein each row comprises an indicator in a column in the first portion of the symptom matrix when a patient-perceived symptom corresponds to one of the possible patient-perceived symptoms, and wherein each row comprises an indicator in a column in the second portion of the symptom matrix when a patient-perceived symptom corresponds to one of the possible ECG events; and
a user interface configured to display the generated symptom matrix.

15. The system of claim 14, wherein the symptom matrix further comprises one or more of: (i) a frequency of the patient-perceived symptoms and/or the possible ECG events experienced by the patient; and (ii) a summary of the patient-perceived symptoms and/or the possible ECG events experienced by the patient.
